(19) United States
(12) Patent Application Publication
(10) Pub. No.: US 2022/0181000 A1
(43) Pub. Date: Jun. 9, 2022

(54) A Surveillance System for a Blood Treatment Apparatus for Monitoring Particular Hygienically Relevant States

(71) Applicant: Fresenius Medical Care Deutschland GmbH, Bad Homburg (DE)

(72) Inventors: Gerhard Wiesen, Bad Homburg (DE); Martin Buehner, Sandberg (DE); Maria Millan-Galante, Bad Homburg (DE)

(21) Appl. No.: 17/440,496

(22) PCT Filed: Mar. 23, 2020
(86) PCT No.: PCT/EP2020/057933

§ 371 Date: Sep. 17, 2021

(30) Foreign Application Priority Data
Mar. 25, 2019 (DE) 102019107540.8

(51) Int. Cl. G16H 20/40; G16H 10/20

(52) U.S. Cl. CPC G16H 20/40; G16H 10/20

(57) ABSTRACT

The present disclosure relates to a system with a blood treatment apparatus and with a surveillance system for monitoring the occurrence of a state of a group of predetermined states, for which states it has been previously predefined or predetermined that when they occur, gloves are to be worn by the user of the blood treatment apparatus in order to avoid contaminations or carrying over of germs.

### CROSS-REFERENCE TO RELATED APPLICATIONS

The present application is the national stage entry of International Patent Application No. PCT/EP2020/057933, filed on Mar. 23, 2020, and claims priority to Application No. 10 2019 107 540.8 filed in the Federal Republic of Germany on Mar. 25, 2019, the disclosures of which are expressly incorporated herein in their entireties by reference thereto.

### TECHNICAL FIELD

The present disclosure relates to blood treatment apparatuses with surveillance systems. Furthermore, the present disclosure relates to digital storage mediums, computer program products, and computer programs.

### BACKGROUND

When treating patients, special attention is paid to avoid infections between patients treated simultaneously or successively in the same clinic or station. If medical devices, e.g., blood treatment apparatuses are used for treating patients, it is important to monitor that these are not a source for infections to patients who are treated in the future or simultaneously.

The present disclosure describes surveillance systems for a medical device, e.g., for a blood treatment apparatus, and systems suitable for monitoring, as well as digital storage mediums, computer program products, and computer programs.

Advantages of the systems and methods described in the present disclosure are achieved by surveillance systems as disclosed herein. Advantages are further achieved by digital storage mediums, computer program products, and computer programs as disclosed herein.

In all of the aforementioned and following statements, the use of the expression “may be” or “may have” and so on, is to be understood synonymously with “preferably is” or “preferably has”, and so on, respectively, and is intended to illustrate an embodiment according to the present invention.

Whenever numerical words are mentioned herein, the person skilled in the art shall understand them as indications of numerical lower limits. Hence, unless this leads to a contradiction evident for the person skilled in the art, the person skilled in the art shall comprehend for example “one” as encompassing “at least one”. This understanding is also equally encompassed by the present invention as the interpretation that a numerical word, for example, “one” may alternatively mean “exactly one”, wherever this is evidently technically possible in the view of the person skilled in the art. Both of these understandings are encompassed by the present invention and apply herein to all used numerical words.

Whenever the terms “programmed” or “configured” are mentioned herein, it is thus disclosed that these terms are interchangeable.

The information “top” and “bottom” are herein to be understood in case of doubt by the person skilled in the art as absolute or relative spatial information, which refers to the orientation of the respective element when used as intended.

The surveillance systems disclosed herein serve for monitoring whether a predetermined state is present, occurs, or is about to occur, during or in association with using a medical device, e.g., a blood treatment apparatus. If this is the case, the surveillance system automatically initiates one or more actions with the aim of contributing to the safety of the patient.

Predetermined states are characterized by the fact that it has been beforehand determined for them—e.g., based on experience, legal regulations, etc.,—ex works, by the clinic, station, or the user of the blood treatment apparatus (mostly medical or nursing staff), that in case they occur, predetermined actions will be taken or effected in order to avoid contaminations, cross contaminations (transmitting germs from one patient to another, or from one blood treatment apparatus to another), or carrying over of germs. For these states, it may apply that when they occur, e.g., gloves are to be worn by the user of the blood treatment apparatus or worn gloves are to be changed or removed. Thus, it may be determined that when such a state occurs, the user has to wear gloves for upcoming activities in association with the blood treatment apparatus or with another medical device or has to remove the gloves, e.g., after an activity by which the user wore gloves.

The predetermined state is defined or described in advance by concrete features as mentioned below. Since a series of states for which wearing gloves is mandatory may occur during treatment, a group of such states, i.e., optionally more than just one such state, is put together and saved in practice.

Monitoring may alternatively be understood herein to mean detecting, further alternatively, indicating or notifying that such a state has occurred or is about to occur, or combinations thereof.

The surveillance system which serves for such monitoring encompasses at least one storage device, detecting device, receiving device, and executing device, respectively suitably programmed or configured. These devices may be embodied by individually present components, or be implemented in any combination in common components.

The group having the at least one predetermined state is stored in the storage device. For each of these states of the group, at least one predetermined signal configuration and at least one predetermined action may be assigned in reaction to a signal having this configuration.

In the storage device, a determination, description, definition, or occurrence condition of the state may be additionally or alternatively stored for each of the states of this group. The terms determination, description, definition, or occurrence condition may, in some embodiments, respectively replace the term “definition” used below, and vice versa.

The predetermined states, which the group encompasses or consists of, may be states (Z₁, . . . , Zₙ) related to the use of a blood treatment apparatus.

The detecting device is configured to detect whether at least one of the states defined in the group has occurred. For this purpose, it may optionally have at least one suitable sensor or may resort to sensor results of present sensors, e.g., of the blood treatment apparatus. The detecting device is further configured to, in the event that it detects that one of the defined states has occurred, send a signal which is associated to the detected, predetermined state, namely in the configuration assigned to that state. The detecting may be, or may include, measuring, comparing, reading, evaluating, and/or calculating.

The receiving device is configured to receive each of the transmitted signals and to prompt, upon receipt thereof, at least one predetermined action assigned to the signal.

The executing device is configured to, when it receives such a signal from the receiving device, execute the action respectively assigned to the transmitted signal in the storage device.

In this, the entire group of states (Z₁, . . . , Zₙ) may also be assigned only one signal, and accordingly also the same action connected thereto, respectively. Likewise, this may apply to a part of the states of the group (Z₁, . . . , Zₙ). The signal-action combinations which are assigned to the individual states Zₓ of the group (Z₁, . . . , Zₙ) do not necessarily have to be different from each other.

According to the present disclosure, a system is further proposed which comprises, in addition to the surveillance system disclosed herein, a medical device, e.g., a blood treatment apparatus.

A storage device (denoted here also as carrier) according to the present disclosure, e.g., a digital storage device, e.g., a non-volatile storage device, for example in the form of a disk, RAM, ROM, CD, hard disk, DVD, USB stick, flash card, SD card, or EPROM, e.g., with electronically or optically readable control signals, programmed to program or configure a blood treatment apparatus, which comprises a storage device, a detecting device, a receiving device and an executing device, into a system according to the present disclosure is likewise proposed.

A computer program product according to the present disclosure comprises a volatile program code or a program code saved on a machine-readable carrier by which a blood treatment apparatus, which comprises a storage device, a detecting device, a receiving device and an executing device, may be programmed or configured into a system according to the present disclosure.

The term “machine-readable carrier”, as used herein, denotes in certain embodiments according to the present disclosure a carrier which contains data or information which is interpretable by software and/or hardware. The carrier may be a data carrier such as a disk, a CD, DVD, a USB stick, a flashcard, an SD card an EPROM and the like.

A computer program according to the present disclosure comprises a program code by which a blood treatment apparatus, which comprises a storage device, a detecting device, a receiving device and an executing device, may be programmed, reprogrammed or configured into a system according to the present disclosure.

According to the present disclosure, a computer program product can be understood as, for example, a computer program which is stored on a data carrier, an embedded system as a comprehensive system with a computer program (e.g., an electronic device with a computer program), a network of computer-implemented computer programs (e.g., a client-server system, a cloud computing system, etc.), or a computer on which a computer program is loaded, executed, saved, or developed.

A computer program according to the present disclosure can be understood as, for example, a physical software product, which is ready for distribution and has a computer program.

Advantageous developments of the present disclosure are subject-matter of the embodiments.

Whenever an embodiment is mentioned herein, it is then a purely exemplary embodiment according to the present disclosure.

Embodiments according to the present disclosure may comprise one or several of the features mentioned supra and/or in the following in any combination, provided the specific embodiment is not recognized by the skilled person as technically impossible.

A predetermined state within the meaning of the present disclosure, when detected by the detecting device, may be a trigger for a signal that the detecting device transmits based on the signal configuration stored for this state in the storage device.

Herein, a “definition” may generally be a description, e.g., of an observation, of a sensor result or of a machine behavior (e.g., treatment progress, read out of control software).

A description may for example be: “user is not wearing gloves”, “user has approached the blood treatment apparatus”, etc. A machine behavior may for example be: the control device or the closed-loop control device (in short: control device) effects that the termination of the blood treatment, that pumps are stopped and/or that the blood treatment apparatus proceeds into reinfusion, that a “blood leak alarm”) has been triggered, that a notification such as “patient must be connected” is displayed.

Each of these definitions may be associated with a predetermined action, such as following an IF-THEN rule (IF for example a blood leak alarm is triggered, THEN for example the notification that gloves must be worn is triggered as well). In connection thereto, it is determined which type of signal it is that triggers the action, namely, e.g., a signal that is transmitted to a monitor and that effects showing this specific, predetermined text as a notification for the user.

Associated with a definition may be that, e.g., a hook or a marker has been set in a software for certain states. Thus, the manufacturer, the technician and/or the user may determine which of the states that may occur when using the blood treatment apparatus, counts as a state of the group mentioned herein.

In some embodiments, the executing device is or encompasses at least one monitor, screen, or display configured for displaying to the user of the treatment apparatus a notification corresponding to the signal. This monitor may be part of the blood treatment apparatus, it may be a monitor separate thereof.

In several embodiments, the executing device is or encompasses a handheld device, which is intended to be held and/or carried by the user. Such handheld device encompasses smartphones, Blackberrys, pagers, beepers, clips, pendants, headsets, smart wristwatches, or wrist-worn devices.

The executing device may comprise the receiving device and is thereby optionally in signal communication with the detecting device.

In some embodiments, the executing device is or encompasses the control device or the closed-loop control device of the blood treatment apparatus or a section thereof. Thus, the control device or closed-loop control device of the blood treatment apparatus may be configured so that, as a predetermined action, upon receiving a signal which is associated to one of the predetermined states of the group of the hygienically relevant states, treatment options, or operational options, etc., are blocked, pumps are stopped, etc. This intervention of the control device or closed-loop control device in the treatment course may continue until the surveillance system detects that the user is wearing gloves, until the user confirms a notification, and/or until another, likewise predetermined, circumstance occurs.

In several embodiments, the action which the executing device prompts upon receiving the signal associated to the predetermined state is or encompasses outputting a predetermined notification on the executing device, e.g., on the monitor or on the display.

Alternatively or additionally, the action is or encompasses prompting a component to vibrate, outputting predetermined color representations, outputting an optical or an acoustic alarm, requiring a confirmation by the user before he or she may continue with the use of the treatment apparatus or with predetermined options of the treatment.

The action may further encompass storing a history. The history may provide information about the time and about how frequently messages, notification or alarms associated to the states of the group have occurred, e.g., stating an importance, relevance, or evaluation. Such a history may serve the further education of the staff and/or the quality control.

In certain embodiments, the following observations or occurrences count as a state:
the surveillance system detects that the user is successively working on two medical devices, e.g., blood treatment apparatuses, which have previously been classified as hygienically relevant;
the surveillance system detects an alarm state (an alarm emitted, e.g., by the density sensor, optical sensor, air detector, pressure detector, blood detector, access sensor, or by another sensor of the blood treatment apparatus);
the surveillance system detects a predetermined operational step at the blood treatment apparatus, such as priming; pending connection of the patient; increasing the flow by the blood pump; changes of a measured pressure; disconnecting the patient; actuating or selecting a switch; stopping a predetermined pump;
the surveillance system detects that the user enters a predetermined zone surrounding the blood treatment apparatus at least in section or that the user is about to enter the zone or that the user leaves or is about to leave the zone.

In some embodiments, the detecting device is a sensor, a software, routine, program loop, or respectively a section of each, a camera, a touchscreen capacity meter, and/or a zone surveillance device.

The following statements indicate embodiments for the implementation of the present disclosure. They relate to the definition of predetermined states, partially with associated actions.

For example, the knowledge that the user must handle himself the blood accesses in or a certain phase of preparing the blood treatment apparatus for the upcoming treatment session or during the blood treatment itself may count as a predetermined state (and correspondingly be saved in the storage device as a state and its occurrence is read out from the operating software by the detecting device).

A state may be detected for example when connecting the patient (the device is, e.g., in the priming mode), when disconnecting the patient (the device is, e.g., in the reinfusion mode or thereafter) or alarm situations by which it is likely that the user has or is about to have contact to the blood hoses/accesses or other hygienically relevant components.

For monitoring the connection of the patient, the surveillance device may be optionally configured as follows: The blood treatment apparatus has optionally a menu item on the GUI (Graphical User Interface), where the phase may either be automatically displayed or selected by the user. Accordingly, the blood treatment apparatus is in the “priming mode” or in a preparation mode, respectively, until it is no longer selected. When detecting the end of this phase by corresponding software, the blood treatment apparatus may have a sensor, such as an optical detector (IR) and/or a density detector (ultrasound) which indicates blood flowing in the arterial line. Therefore, the detector may optionally determine at the arterial or venous hose the properties of the fluid present therein. Alternatively or additionally, a starting/increasing of the blood flow (corresponding to a set value or a directly/indirectly measured flow) over a limit may be also used as criterion, since it is not to be expected that the user will increase the flow prior to a clean connection of lines. Therefore, also pressure value in the tube system may be used, since this of course depends on the blood flow (rotation of the pump). Forwarding the knowledge that one of these states has occurred requiring an action may be transmitted by the software (which is, e.g., provided for regulating the blood treatment apparatus) via signal to the detecting or receiving device.

For monitoring the reinfusion of blood after the treatment, the surveillance device, by way of example, may be configured so that the surveillance system detects that a treatment has been completed, for example, by the blood treatment apparatus detecting that a prescribed ultrafiltration amount has been withdrawn from the blood or that a predetermined treatment time has elapsed. For this purpose, the surveillance system may be configured as follows: The blood treatment apparatus may have a menu item on the monitor, e.g., on the GUI (Graphical User Interface). There, the phase (reinfusion phase) may either be automatically displayed or selected by the user. If the blood treatment apparatus passes into one of these phases due to a user input or automatically, this may be predetermined as a state for which an action and thus also a signal triggered by this action are stored in the storage device. The action may be to give a notification that gloves are to be worn for upcoming activities or actions using the blood treatment apparatus. Alternatively or additionally, other or further actions are also conceivable.

For monitoring the disconnecting of the patient, for instance after the so-called reinfusion phase or after an uncoupling phase subsequent to the reinfusion phase in which the patient is disconnected and/or bags etc. are uncoupled, the surveillance system is optionally configured as follows: The blood treatment apparatus may have a menu item on the monitor, e.g., on the GUI (Graphical User Interface). There, the phase (e.g., uncoupling phase) is either automatically displayed or may remain to be selected by the user. If the blood treatment apparatus passes into one of these phases due to a user input or automatically, this may be predetermined as a state for which an action and, thus, also a signal triggered by this action are stored in the storage device. The action may be to give a notification that gloves are to be worn for the upcoming activities at the blood treatment apparatus. Alternatively or additionally, other or further actions are also conceivable.

For an automatic start of this phase, the blood treatment apparatus may have a sensor, for example an optical detector (e.g., IR) a density detector (e.g., ultrasound) etc., which displays when no or substantially no blood is flowing in the venous line. Therefore, the detector may detect at the venous hose the properties of the fluid present therein. Alternatively, it is also possible to use a pressure value in the hose system, since at the end of the reinfusion phase the blood pump is stopped and pressure compensation takes place in the entire fluid system.

Alternatively or additionally, it is possible to detect that a predetermined treatment time has elapsed. This too may be stored as a predetermined state; it is hereby also assumed that next the user will disconnect the patient from the patient lines and, e.g., take care of the puncture sites.

For example, it may be programmed so as not to assume an upcoming disconnection of the patient, as long as the optical sensor, e.g., for optical density, does not display a change from “dark” to “bright”. Thus, it may be provided that no message is sent to the user and/or no state for this case is saved stating that there is no recognizable change of the fluid flowing through the extracorporeal blood circuit (i.e., also no detected change of the optical density from “bright” to “dark” or from “dark” to “bright”), meaning that there is no blood displaced by transparent rinsing liquid towards the venous blood vessel needle of the patient, and/or no fluid below a specified density etc. is displayed.

Furthermore, predetermined states may be stored, upon which occurrence it is likely that the user has or will have contact to blood hoses/accesses.

In some embodiments, it is provided to undo or to terminate an already prompted action in case the state should not last. Thus, it may be programmed in the event of an alarm being a predetermined state, a notification already displayed on the monitor (e.g., via the GUI) according to which, for example, gloves are to be worn, changed, or taken off, should not be further displayed. Since the alarm state, which is considered to be a predetermined state, no longer applies, a corresponding notification or other action may no longer be required. Similarly, it may apply that when, e.g., a pump which has been stopped due to an alarm state is restarted, then the alarm state no longer exists.

A further predetermined state may occur when a negative arterial pressure alarm (indicating that the needle is sucking in the vessel) is detected. The surveillance system may optionally be configured as follows: The device has a so-called arterial pressure sensor. The latter is arranged to measure the pressure in the area of the hose near the arterial needle (for example, between needle and blood pump). If the pressure drops below or to a predetermined limit, this will be interpreted that there is a problem with the arterial access. In order to remedy this error status, it is to be expected that the user comes into contact with the vascular access or with the hoses.

Yet another predetermined state may occur when a “venous access monitoring alarm” occurs (indicating loss of the venous needle). The surveillance system may optionally be configured as follows: The device has a so-called venous pressure sensor. The latter is arranged to measure the pressure in the area of the hose near the venous needle (e.g., at a venous blood chamber). If by this pressure a disconnection of the needle or a release of the needle/hose connection is assessed, this pressure falls below or by a predetermined value, as it is, e.g., the subject-matter of WO 2009/118145 A1. Alternatively, other methods for monitoring a needle disconnection and their alarm signals may be used a trigger. Such methods may be moisture sensors at the puncture site, as it is, e.g., the subject-matter of WO 2011/116943 A1, optical methods, as it is, e.g., the subject-matter of WO 2008/123814 A1 or electrical methods, as it is, e.g., the subject-matter of WO 2003/086505 A1. In order to remedy this error status, it is to be expected that the user comes in contact with the vascular access or with the hoses. The disclosures of the aforementioned documents are hereby incorporated by reference in their entirety as the subject-matter of this disclosure too. They may be considered as examples of the description of predetermined states of the group.

Yet a further predetermined state may occur when a blood leak detector alarm occurs. The surveillance system may optionally be configured as follows: the blood treatment apparatus has a so-called blood leak detector. This detector is a detector that is arranged downstream of the dialyzer or of the blood filter at the dialysate outlet line and may already detect small amounts of blood. This is usually done optically. The detecting device of the surveillance system according to the present disclosure may be configured to emit a corresponding signal upon detecting a blood leak, upon receiving the signal the executing device triggers or prompts an action (notification, alarm, etc.) associated with the case of a blood leak.

Yet another predetermined state may occur when an air alarm/microbubble alarm occurs. This may require that the user disconnects the patient to remove the air from the system. The surveillance system may optionally be configured as follows: The blood treatment apparatus optionally has one or several so-called air detectors. These are detectors that are arranged at a blood-bearing line. This usually takes place by ultrasound, however may also be done by electrical or optical methods. Upon detecting something, it may then be necessary for the user to release air bubbles, for example by moving the tubes, or even disconnect the patient. It may be saved for this case that hereby it is required to wear gloves. The air alarm/microbubble alarm may be defined as a predetermined state which triggers a previously defined signal and an associated action thereto, as saved in the storage device.

Yet a further predetermined state may occur when entering into or leaving a predetermined zone (e.g., surrounding the blood treatment apparatus) by the user or generally by any person is detected. Such a state is comparatively easy to detect by methods which allow a spatial resolution in order to detect persons present there. Such methods may encompass or be based on, e.g., triangulation (with WLAN, IR, RFID), temperature sensors, cameras, motion detectors, etc.

When put into practice, the necessary apparatuses and devices (sensors, cameras, etc.) do not necessarily have to be installed on the device, they may also be disposed elsewhere in the space to be captured or recorded.

For example, a space comprising 1 m width surrounding the blood treatment apparatus may be defined as a zone whose entry (or exit, since the user may have gone to the next patient after leaving the zone) may be saved as a predetermined state and detecting it may trigger a corresponding signal.

The defined zone may be symmetrical. It may be asymmetrical. It may, for example, have 1 m towards the patient bed, but only 40 cm towards an adjacently arranged blood treatment apparatus. Markings (optionally removable) may be used in the space in order to define the zones.

It is advantageously not pointed out by the present disclosure, always and regardless of the pending activities of the user, to change or wear gloves, rather only when one of the predetermined states and thus previously determined as particularly hygienically relevant occurs. The restriction to the particularly hygienically relevant states may advantageously a) obviate the obtunding of the user to notifications, alarms, or other states related to the use of gloves, and b) possibly decrease the consumption of (not necessary) gloves.

In some circumstances, it may be necessary to a priori ensure that the user wears gloves, possibly regardless of what activities are to be directly performed by him or her. Such circumstances may be, e.g., dealing with known infectious patients, patients with known colonization by multi-resistant germs, etc.

It may be desired for such, or for other, cases to check whether the user is wearing gloves. The same applies, if one wishes to check whether the user has followed, e.g., a request, transmitted via notification, to wear gloves.

For such cases, there may be saved a, possibly further, predetermined state that already occurs when the surveillance system detects that the user is not wearing gloves.

Failure to wear the gloves may be detected by suitable sensors, e.g., optical sensors, capacity measuring sensors, heat sensors, etc. which are connected to, or are part of, the detecting device.

Thus, one may detect, e.g., using a touch screen, whether the user is wearing gloves. This happens, for example, by the current flowing through the screen or by a capacitive signal. The use of the touch screen may vary measurably, depending on whether the user wears gloves during use or not.

If the user is not wearing any gloves, any action referred to herein may be considered as an action to be takes or initiated upon receiving the signal by the executing device.

Thus, for example using the blood treatment apparatus, for instance confirming or terminating an alarm or a notification, or already starting the blood treatment, selecting a treatment option, connecting lines, etc. would then be impossible if the surveillance system detected that the user is not wearing any gloves.

The detecting device may be part of the blood treatment apparatus, it may however also be part of a further apparatus of the surveillance system or it may be a separate element.

The detecting device is, or comprises, a sender (Bluetooth, WLAN, etc.) with which it may directly or indirectly communicate with the receiving device, e.g., a receiver on the wristband.

In several embodiments, the detecting device is located in the room (patient room, treatment room etc.) or in the building (clinic, station, dialysis department, etc.).

The detecting device is in several embodiments designed to communicate with the receiving device wirelessly or via a wired connection.

If the detecting device is, e.g., part of the blood treatment apparatus and/or falls back to its components, then this may advantageously contribute to the fact that the present disclosure may be implemented in or at blood treatment apparatuses already in-use. Thus, it may be sufficient to update the operating software of the blood treatment apparatus by, e.g., a computer program according to the present disclosure which is cost-effective and easy.

This may be the case, e.g., when the predetermined state is a machine behavior of the blood treatment apparatus in the course of the blood treatment, for instance the transition into the reinfusion or the like, or when the predetermined state is an alarm state of the blood treatment apparatus, and when additionally the executing device is the monitor of the blood treatment apparatus which outputs corresponding messages like “wear gloves” as action to the user.

The executing device is in several embodiments an alarm device.

The executing device is in several embodiments a monitor or comprises same.

The executing device is in several embodiments wearable on the body, e.g., by a wristband and thus close to the glove and/or well visible and/or operable, for instance for confirming a notification on a monitor or for turning off a vibrating alarm or an acoustic alarm. Additionally or alternatively, the executing device may be a clip, a pendant, etc.

The executing device is in several embodiments configured to generate a vibrating alarm, an optical, and/or an acoustic alarm.

An optical alarm may be color-coded (e.g., two colors, e.g., red and green, wherein red may indicate that the user has something to do). A color-coding as, e.g., the use of red light, or light in different colors, may, as other alarm types—with a possible exception of the pure vibrating alarm—be recognized by an outsider (i.e., a person other than the responsible user).

Other signals, for example different shades of brightness, interrupted light versus continuous light, sound (volume, frequency), etc. are also possible according to the present invention

The executing device is in several embodiments configured to output further information, in addition to the aforementioned alarm related to wearing, carrying, changing, and/or removing gloves, for example by the monitor or display which the user wears, e.g., on the wristband. This further information may include, e.g., alarms that are associated with other medical devices.

The executing device may be part of a further medical device, for instance of a further blood treatment apparatus. Thus, it may be known which device the user will operate next.

In several embodiments, the executing device is configured to, upon confirmation of a notification that, e.g., gloves are to be put one, worn, changed, or removed, visibly terminate an alarm for third parties (patients, doctors, other nursing staff), in that, e.g., a color marking changes similar to a traffic light from “red” to “green”. In this way, it will be possible also for third parties to detect that hygiene measures are required or have been carried out, respectively. Controlling the user by third parties is thereby advantageously possible. Thus, even the patient may point out the necessary hygiene measures or the hygiene protocol to the stressed or inexperienced user, which may increase patient safety.

In several embodiments, the surveillance system is configured to save the history of the surveillance system behavior, e.g., of its detecting device or its executing device. Thus, to stay with the previous example, e.g., the information how often the color marking was red, or how long it was red or any other type of action may be saved, for instance in the storage device. A reading out of this saving may then be used, for example, to increase the patient safety, in that the user may be addressed or further trained based on the saved data with regard to the hygiene protocol. The storage device used for this purpose may be part of the blood treatment apparatus, part of a server of the clinic, station or the like or may be a separate storage device.

Advantageously, the history may also be used to assign specific incidents to specific persons. For this purpose, it is conceivable that the blood treatment apparatus recognizes the respective user and may assign his or her alarms to him or her. In order to do that, there are several conceivable methods: a) the user must identify himself to the blood treatment apparatus, e.g., through actively inserting, e.g., a smart card; b) the user is automatically recognized by the blood treatment machine, e.g., by RFID, NFC chip, tags, GPS transmitter, mobile phone tracking, barcode recognition, face recognition, or the like.

Identification devices may be integrated into the blood treatment apparatus or configured to communicate with the latter. As an example, a facial recognition by camera in combination with a device identification is mentioned here. This may be particularly relevant because it may contribute to a better monitoring of the staff and thus to quality assurance, especially in the hectic alarm situation.

In several embodiments, a device of the surveillance system, e.g., the detecting device or the executing device is configured so that when the surveillance system detects that the user is leaving the zone, an action is only to be done, e.g., a notification is only to be outputted, when it has been detected that, in connection with an executed activity performed on the blood treatment apparatus whose surrounding or zone the user is about to leave, gloves were worn thus requiring that the gloves are now, e.g., to be removed or to be changed. This may be detected in any manner described herein, e.g., by the touch screen of the blood treatment apparatus which zone the user is about to leave.

In several embodiments, a device of the surveillance system, e.g., the detecting device or the executing device is configured as follows: When the surveillance system detects that the user has performed an activity or an action at a first medical device for which the wearing of gloves is mandatory, the surveillance system however fails to detect simultaneously that the user has worn gloves when performing the activity at the first medical device, then a corresponding action is carried out by the surveillance system. For example, it may be pointed out that gloves must be worn and/or hands must be disinfected.

In this, the surveillance system may additionally be configured such that the action is executed only when according to a saved work plan, a second blood treatment apparatus will have to be operated and/or when it is detected by a recognizing device, e.g., a camera or a detecting device as described supra that a predetermined area surrounding a second blood treatment apparatus is entered.

A treatment apparatus according to the present disclosure may be suitable and/or configured for performing hemodialysis, hemofiltration, hemodiafiltration, or a separation method, without being limited thereto.

In some embodiments, the disclosure is not directed to a blood treatment apparatus, rather to a medical device by the use of which it is likewise expected that the user of the device wears gloves. Such devices may likewise be treatment apparatuses, but they may also be dialysis apparatuses or serve a different purpose. For theses embodiments, what is described herein for blood treatment apparatus applies analogously to the medical device. Thus, in these embodiments, the terms “blood treatment apparatus” and “medical device” may be interchangeable.

In some embodiments, no disinfection state, e.g., of a touch screen (or of a touch screen sensor), is monitored or captured. No signal is emitted indicating the disinfection state of the touch screen (or of a touch screen sensor). The present disclosure does not relate to, nor encompasses, any system or apparatus which would be programmed to monitor the disinfection state and/or to emit such a signal.

In some embodiments, no disinfection state vector is created. The present disclosure does not relate to, nor encompass, any system or apparatus which would be programmed to create a disinfection state vector.

Disinfecting a touchscreen, a touch screen sensor, or a touch screen capacitance meter is not provided in some embodiments. The present disclosure does not relate to, nor encompass, any system or apparatus which would be programmed to disinfect the touchscreen, touch screen sensor, or touch screen capacity meter.

One or several advantages mentioned herein may be achieved by several embodiments.

An advantage of the present disclosure is that it is very easy to implement. Thus, it may be possible to retrofit a conventional blood treatment apparatus to a blood treatment apparatus according to the present disclosure only by a software update. Also, outputting notifications or glove alarms, for instance on the smart phone carried in the pocket of the user or on the room alarm or machine alarm, by vibrating alarm, alarm tone, ringing tone reserved for this purpose may be realized without significant technical effort.

The present disclosure may contribute to advantageously prevent carrying over of germs between patients or between blood treatment apparatuses or may at least reduce the risk thereof. This advantage should come into effect especially with still inexperienced users.

Moreover, the present disclosure allows for different hygiene protocols within a clinic or station and even within a patient room with more than one patient. Thus, it may be provided: allowing a first monitoring device for monitoring a first patient (or a first blood treatment apparatus) to access a first group of states; allowing a second monitoring device for monitoring a second patient (or a second blood treatment apparatus) to access a second group of states. Both the first blood treatment apparatus and the second blood treatment apparatus may be used in the same station or in the same patient room. If the states which are respectively accessed by their executing devices are different from each other, they will then instruct the users of these two blood treatment apparatuses to behave differently in relation to the use of gloves. Using the systems as disclosed herein in this way may above all offer advantages when, for instance, with regard to possible carrying over of germs, uncritical patients are treated together with very critical patients, with regard to their germ burden (MRSA or other multi-resistant germs), in one room, in one station, etc.

Simultaneously, the present disclosure may contribute to reducing the consumption of gloves to a reasonable level, if the states are respectively selected appropriately.

### BRIEF DESCRIPTION OF THE FIGURES

The present invention is exemplarily explained with regard to the accompanying drawings in which identical reference numerals refer to same or similar components. The following applies in the figures:

FIG. 1 shows schematically simplified sections of a blood treatment apparatus with a surveillance device in an exemplary embodiment;

FIG. 2 shows a surveillance system roughly simplified as a diagram; and

FIG. 3a bis 3c show examples of the occurrence of a predetermined state.

### DETAILED DESCRIPTION

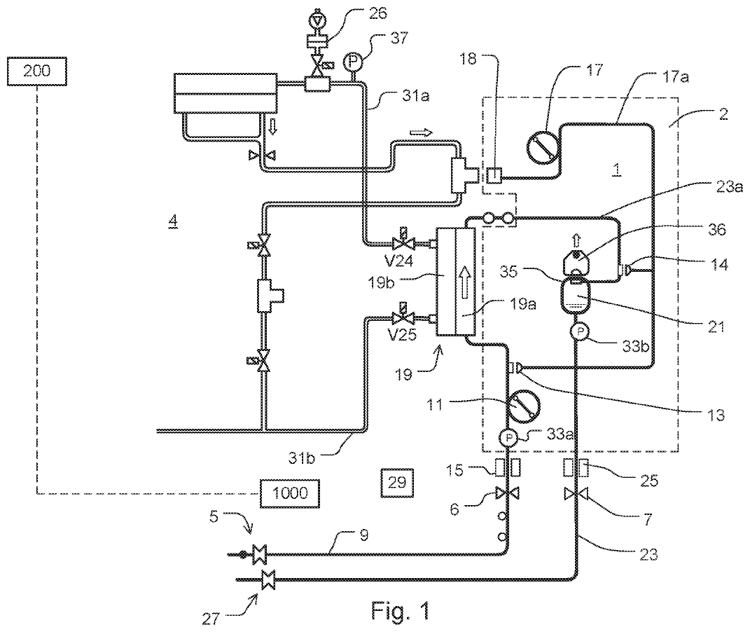

FIG. 1 shows an extracorporeal blood circuit 1, which would be connected for a treatment to the vascular system of the patient (not shown) via double-needle access or via single-needle access using, e.g., an additional Y-connector. The blood circuit 1 is provided optionally in sections thereof in or on a blood cassette 2. This blood cassette 2 is optionally designed to be used also with other treatment types, e.g., a single-needle treatment.

Pumps, actuators, and/or valves in the area of the blood circuit 1 are connected to a blood treatment apparatus 4 or a control device and/or closed-loop control device 29, which comprises, or is in signal communication with, a surveillance system 1000, or parts thereof. The surveillance system 1000 encompasses a detecting device 200 or is connected to same in signal communication. The detecting device 200 may exemplarily be a camera.

The blood circuit 1 comprises an arterial patient hose clamp 6 and an arterial connection needle 5 (as an example for an access device) of an arterial section or of an arterial patient line or blood withdrawal line 9. The blood circuit 1 further comprises a venous patient hose clamp 7 and a venous connection needle 27 (as an example for a further or second access device) of a venous section or of a venous patient line or blood return line 23.

A blood pump 11 is provided in the arterial section 9, a substituate pump 17 is connected to a substituate line 17a. The substituate line 17a can be connected with a substituate source through a, preferably automatic, substituate port 18. By the substituate pump 17, substituate may be introduced via pre-dilution or via post-dilution through associated lines 13 or 14 into line sections, e.g., into the arterial section 9 or into a venous section 23a (between a blood chamber 19a of a blood filter 19 and a venous air separation chamber 21), of the blood circuit 1.

The blood filter 19 comprises the blood chamber 19a which is connected to the arterial section 9 and to the venous section 23a. A dialysate chamber 19b of the blood filter 19 is connected to a dialysis liquid inlet line 31a which is leading to the dialysate chamber 19b and to a dialysate outlet line 31b which is leading away from the dialysate chamber 19b.

The dialysis liquid inlet line 31a optionally comprises a valve V24 by which the flow within the dialysis liquid inlet line 31a may be blocked. The dialysate outlet line 31b optionally comprises a valve V25 by which the flow within the dialysate outlet line 31b may be blocked.

The dialysis liquid inlet line 31a is further optionally connected to a compressed air source 26 by another internal valve of the machine. The compressed air source 26 may be provided as a component of the treatment apparatus 4 or as a part separate thereof. A pressure sensor 37 may be provided downstream of the compressed air source 26.

FIG. 1 further shows an arterial air/blood detector 15, a venous substituate/blood detector 25 and pressure sensors 33a, b. They can be used by the detecting device (200).

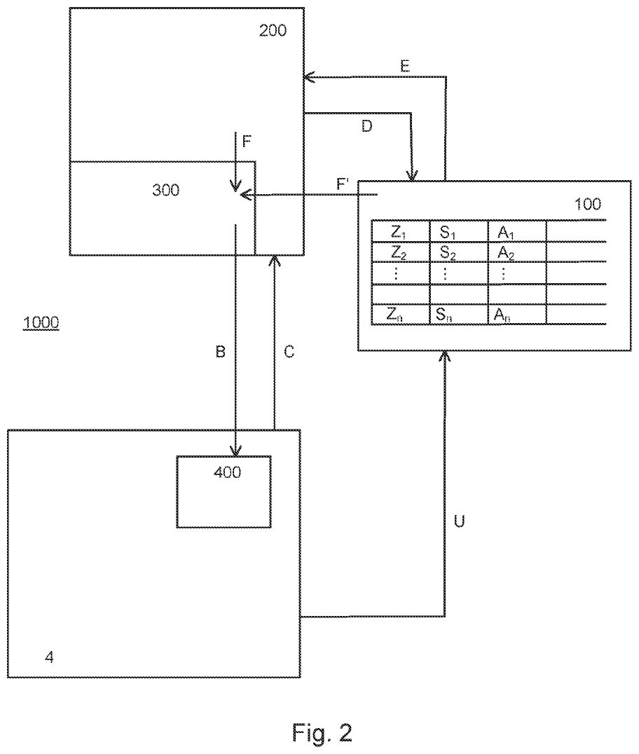

FIG. 2 shows a surveillance system 1000 for monitoring the occurrence of a certain state (Z₁, . . . , Zₙ) in a roughly simplified manner as a diagram. The arrows (labeled B to F, F′ and U) indicate data flows which are necessary for the above-mentioned monitoring by the surveillance system 1000. The data may each be transmitted by electronic connection, cable or wireless.

Dividing the individual devices in 100, 200, 300, 400 is purely exemplary. They may also be respectively individually or multiply integrated into the blood treatment apparatus 4 or another medical device. Likewise, any connected combinations of the single devices 100, 200, 300, 400 in separate housings are conceivable or as well each device individually. It is further contemplated to provide some or all of the devices 100, 200, 300, 400 as software implementations.

The surveillance system 1000 encompasses a storage device 100, in which there are definitions or descriptions of previously defined hygienically relevant states Z₁, . . . , Zₙ, here exemplarily in tabular form. Each state Zₓ (x=1, . . . , n) has at least one corresponding signal Sₓ assigned thereto in the respective table line and at least one respectively corresponding action Aₓ to the signal Sₓ. It is possible to supplement the table with further data according to the states. It is also conceivable to update, modify and/or extend, e.g., also in order save a history as discussed supra, the data of this table by the data flow A from the blood treatment apparatus 4.

The surveillance system 1000 further comprises a detecting device 200 which, based on the data available by the data flow C, e.g., sensor data or treatment data of the blood treatment apparatus 4, including the states saved in the storage device 100 by most different operations, as discussed above, may determine whether a hygiene-relevant state Zₓ from the group Z₁, . . . , Zₙ occurred. In the simplest case, this is done by comparing the saved states Zₓ (x=1, . . . , n) to an actual situation.

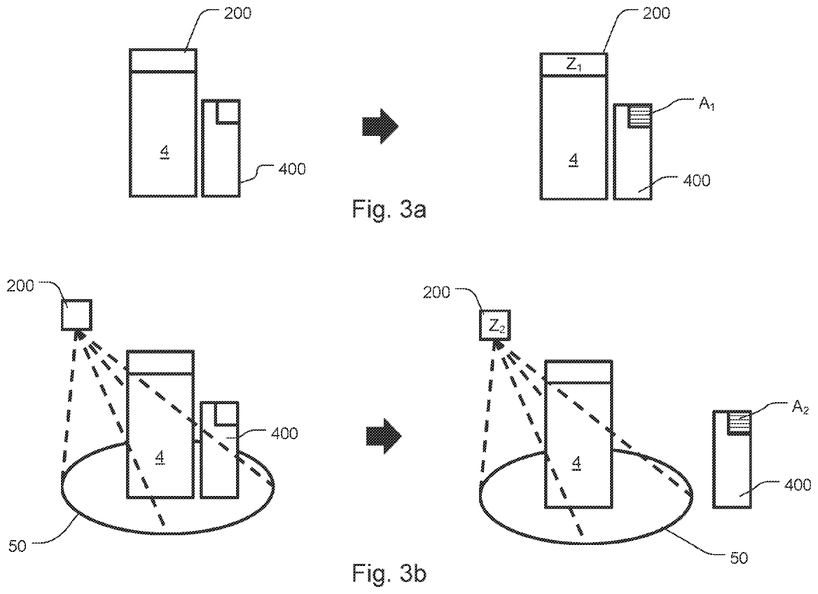

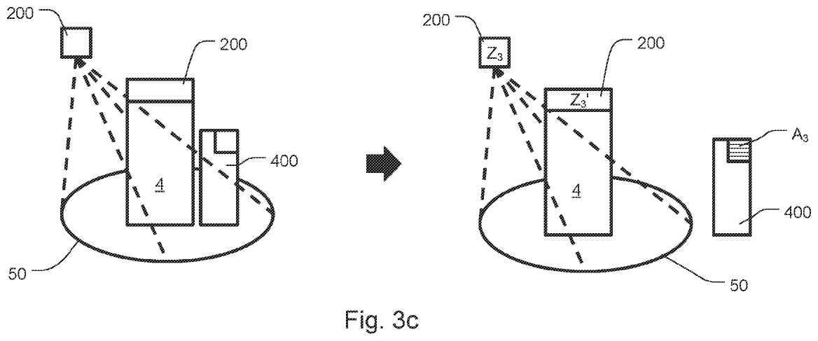

If this is the case, then the detection device 200 transmits via the data flow F the signal Sₓ assigned to the determined state Zₓ to a receiving device 300. The latter then forwards (via the data flow 3) the action Aₓ to be performed, which is assigned to the signal and was read out here via the data flow F′, using an executing device 400, here for example a display or another communication device of the blood treatment apparatus 4, a wristband, a mobile phone, a tablet, a pager, a beeper, etc., for the information of the user. FIG. 3a shows an example of the occurrence of a predetermined state Z₁ and the interaction of the blood treatment apparatus 4 at which the state Z₁ occurs, with a handheld device as an example of an executing device 400.

In the example of FIG. 3a, the blood treatment apparatus 4 comprises at least the detecting device 200 and optionally also further components of the surveillance system 1000 mentioned herein.

By contrast, the executing device 400 is in the example of the FIG. 3a a handheld device that the nurse carries with her or him.

In the left-hand illustration of FIG. 3a, no state belonging to the group of the predetermined states Zₓ is detected by the detecting device 200. Therefore, no signal is emitted, that would lead to the predetermined action.

However, this changes shortly afterwards, which is indicated by the arrow between the left and right illustration of FIG. 3a.

In the right-hand illustration it can be seen that the detecting device 200 has now detected the state Z₁ of the group of the predetermined states. This state may be for example a blood leak alarm, for which there is saved that when reacting to the alarm the nursing staff should wear fresh gloves. As a result of the occurrence of this state, e.g., a notification is issued on the handheld device of the nursing staff as an example of an action A₁ assigned to the state Z₁, for example that the gloves must be changed. FIG. 3b shows a further example of the occurrence of a further predetermined state Z₂ and the interaction of the blood treatment apparatus 4, at which the state Z₂ occurs, again with a handheld device as an example of an executing device 400.

Unlike in the example of FIG. 3a, the blood treatment apparatus 4 does not comprise at least the detecting device 200 of the surveillance system 1000. The detecting device 200 is rather a device external to the blood treatment apparatus 4, here exemplarily a camera or a zone surveillance device. It may, as indicated in FIG. 3b, be arranged above the blood treatment apparatus 4 and/or above a patient's bed besides the blood treatment apparatus 4 and for example may monitor a zone 50, indicated by the dashed lines and the circle, according to predetermined criteria for the occurrence of one of the predetermined states.

The predetermined state Z₂, which the camera has detected in the transition of the left illustration to the right illustration of FIG. 3b, is that the nurse is leaving or has left the predetermined, monitored zone 50.

If the predetermined state Z₂ occurs, then an assigned action A₂ e.g. a notification that the previously worn gloves must be changed or discarded, may be issued to the handheld device of the nursing staff.

FIG. 3c shows again another example of the occurrence of a further predetermined state Z₃ and the interaction of the blood treatment apparatus 4, at which the state Z₃ occurs, again with a handheld device as an example of an executing device 400.

Unlike in the examples of FIG. 3a or FIG. 3b, the blood treatment apparatus 4 comprises only a part of the detecting device 200 of the surveillance system 1000. The detecting device 200 is herein to be understood as a combination of devices which, for example, belong to the blood treatment apparatus 4 and devices which are external to the latter, here exemplarily as the camera or zone surveillance device of FIG. 3b.

In order to be able to detect that a predetermined state occurs, the detecting device 200, thus divided, must recognize both a partial state Z₃ by the zone surveillance device and a further partial state Z₃′ by the blood treatment apparatus 4. Only when both partial states are considered to have occurred, the nursing staff receives a notification or the like, which is herein referred to as assigned action A₃. This may, purely exemplary, correspond to the above-mentioned action A₂, which, however, is required only when there has been detected at the treatment apparatus 4 e.g., that the vascular access of the patient was placed (partial state Z₃′) and the nurse has subsequently left the zone 50 or wanted to leave it (partial state Z₃).

Actions may thus be understood as a response to a combination of states, all of which must occur in order to trigger the concrete action.

Likewise, individual states may lead to a combination of actions. Thus, for example upon detecting a blood leak alarm, not only a notification will be sent to the handheld device (as a first action of the combination of actions) but also an alarm sound will be heard on the blood treatment device (as a second action of the combination of actions).

### LIST OF REFERENCE NUMERALS

1 extracorporeal blood circuit
2 blood cassette
4 blood treatment apparatus
5 access device e.g. an arterial connection needle
6 arterial patient hose clamp
7 venous patient hose clamp
9 arterial section or arterial patient line or arterial blood withdrawal line
11 blood pump
13 addition site for substituate (predilution)
14 addition site for substituate (postdilution)
15 arterial air/blood detector
17 conveying device, e.g. substituate pump
17a substituate line
18 automatic substituate port
19 blood filter
19a blood chamber
19b dialysate chamber
21 venous separation chamber
23 venous section or venous patient line or venous blood return line
23a venous section
25 venous substituate/blood detector
26 compressed air source
27 access device for example a venous connection needle
29 control device or closed-loop control device
31a dialysis liquid inlet line
31b dialysate outlet line
33a pressure sensor
33b pressure sensor
35 single-needle valve
36 single-needle chamber
37 pressure sensor
50 zone
100 storage device
200 detecting device
300 receiving device
400 executing device
1000 surveillance system
Z₁, . . . , Zₙ hygienically relevant states or conditions
S₁, . . . , Sₙ signal designs or types
A₁, . . . , Aₙ actions
B data flow
C data flow
D data flow
E data flow
F, F′ data flow
U data flow
V24 valve
V25 valve

1-10. (canceled)

11. A surveillance system for monitoring the occurrence of a state from a group of one or more predetermined states related to the use of a blood treatment apparatus, for which states it has been predefined that when they occur the surveillance system will initiate predetermined actions with an aim of avoiding contamination, cross-contamination, or carrying over of germs, wherein the surveillance system comprises:
a storage device, in which the group of the predetermined states is saved, and in which there is for each of these states at least one predetermined signal and at least one predetermined action assigned as a reaction to the predetermined signal;
a detecting device configured for detecting if at least one of the states included in the group has occurred, and further configured so that, if at least one of the states included in the group has occurred, the detecting device transmits the predetermined signal which is assigned to the detected state of said group;
a receiving device configured for receiving the transmitted predetermined signal and for prompting a predetermined action by an executing device; and
the executing device, configured for executing the predetermined action assigned to the transmitted predetermined signal upon receipt of the transmitted predetermined signal.

12. The surveillance system according to claim 11, wherein the executing device is or comprises:
a monitor, screen, or display for showing a user of the blood treatment apparatus a notification corresponding to the predetermined signal;
a handheld device, which is intended to be held and/or carried by the user; and/or
a control device or a closed-loop control device of the blood treatment apparatus.

13. The surveillance system according to claim 11, wherein the predetermined action is one or a plurality of the following actions:
issuing a predetermined notification for the user on a monitor, screen, or display;
triggering a vibration alarm, an optical alarm, and/or an acoustic alarm;
issuing a notification in color representation;
requesting a confirmation from the user; and
storing a history representing the times and/or how frequently messages, notifications or alarms associated with states of the group have occurred.

14. The surveillance system according to claim 13, wherein issuing a notification in color representation comprises issuing a notification in multicolor representation.

15. The surveillance system according to claim 11, wherein a state applies to one or more of the following states:
the surveillance system detects that the user leaves a predetermined zone which surrounds, at least in section, the blood treatment apparatus;
the surveillance system detects that the user is working or will work in succession, firstly with a first medical device and then with a second medical device which have been previously classified as hygienically relevant;
the surveillance system detects a defined alarm state;
the surveillance system detects a defined operational step on the blood treatment apparatus;
the surveillance system detects that the user is not wearing any gloves; and
the surveillance system detects the entering of the user into a predetermined zone surrounding, at least in section, the blood treatment apparatus.

16. The surveillance system according to claim 11, wherein the detecting device is or encompasses:
a software or sections thereof;
a camera;
a sensor;
a touchscreen-capacity meter; and/or
a zone surveillance device.

17. A system having a blood treatment apparatus and a surveillance system according to claim 11.

18. The system according to claim 17, wherein the blood treatment apparatus is configured for executing a hemodialysis, a hemofiltration, a hemodiafiltration, or a separation method.

19. A digital storage medium with electronically readable control signals, configured for configuring a blood treatment apparatus, the latter comprising, or being in signal communication with, a storage device, a detecting device, a receiving device and an executing device, into a system according to claim 17.

20. The digital storage medium according to claim 19, wherein the digital storage medium is one of: a floppy disk, CD, DVD, or EPROM.

21. A computer program product with a program code stored on a machine-readable medium, configured for configuring a blood treatment apparatus, the latter comprising, or being in signal communication with, a storage device, a detecting device, a receiving device and an executing device, into a system according to claim 17.

22. A computer program with a computer program code for configuring a blood treatment apparatus, the latter comprising, or being in signal communication with, a storage device, a detecting device, a receiving device, and an executing device, into a system according to claim 17.
